(19) United States
(12) Patent Application Publication
(10) Pub. No.: US 2009/0029942 A1
(43) Pub. Date: Jan. 29, 2009

(54) ADMINISTRATION OF COPPER TO AN ANIMAL

(76) Inventors: WAYNE LEECH, Manukau City (NZ); DONALD GEORGE MCLAREN, Aucklannd (NZ); KEITH DAVID MCSPORRAN, Auckland (NZ)

Correspondence Address: FISH & RICHARDSON PC, P.O. BOX 1022, MINNEAPOLIS, MN, 55440-1022, US

(21) Appl. No.: 12/186,147

(22) Filed: Aug. 5, 2008

Related U.S. Application Data
(63) Continuation of application No. 10/474,558, filed on May 17, 2004, now abandoned.

(30) Foreign Application Priority Data
Apr. 9, 2001 (NZ) 509877

(51) Int. Cl. A61K 31/721; A61P 43/00

(52) U.S. Cl. 514/59

(57) ABSTRACT

The present invention is directed to the administration of copper to an animal using a composition containing copper dextran. A method is described for increasing the copper levels in an animal by the administration of a composition containing copper dextran. A preferred method of administration is intra muscularly. The use of this composition has been found to both address copper deficiencies in an animal, particularly deer.

### TECHNICAL FIELD

The present invention is directed to the administration of copper to an animal using a composition that contains copper dextran.

### BACKGROUND ART

A number of an animal suffer from copper deficiencies to the extent that they require the supplemental administration of copper compounds. The causes for such deficiencies may be varied, including being as a consequence of diet, or the predisposition of certain breeds of an animal towards copper deficiencies. In many instances supplements are required.

For example, deer have a requirement for copper, which is often not met by their diet Copper is therefore administered in various forms including injections, oral needles (which lodge within the digestive system for slow release) and drenches. These are administered to prevent the development of nutritional diseases such as osteochondrosis, enzootic ataxia, poor growth, and ill thrift.

Deer are not the only an animal which suffer from nutritional copper deficiencies, though they make a useful case study, as they tend to be more sensitive to the administered copper compounds of the prior art. In particular, deer have a tendency to react adversely to injectable formulations with a high likelihood of local tissue reaction, which may be severe and lead to abscess formation.

In the art both glycinate and edtate compounds of copper are used for addressing copper deficiencies in an animal, and in particular cattle. These are generally introduced parenterally. Both compounds occasionally lead to abscess formation in cattle, though quite commonly to abscess formation in deer. This apparent increased sensitivity in deer represents a significant problem for the administration of parentally administered copper remedies and thus it is one object of the present invention to seek to provide an administrable copper composition, and method for its administration, which can address and/or ameliorate the problems associated with at least glycinate and edtate compounds.

A further complication associated with deer is the relative difficulty of administering oral remedies. This form of administration is common for sheep and cattle, though it is particularly difficult to orally administer any device or composition to deer. Accordingly, most administered compositions for deer are administered parenterally, and thus it would be useful if a composition suitable for parenteral administration to deer were available.

Further, deer typically have a relatively shallow sub-dermal fat layer. As a consequence it is difficult to administer sub-cutaneously but non-intramuscularly. Accordingly it would also be desirable to provide a composition, which was suitable for intramuscular administration to an animal such as deer.

Accordingly, one object of the present invention is to address various problems with the prior art, and to at least provide the public with a useful alternative to prior art compounds such as copper glycinate and copper edtate. At the very least it is an object of the present invention to provide a useful alternative which can be considered in instances where problems may be encountered with the prior art compounds referenced above.

It is an object of the present invention to address the foregoing problems or at least to provide the public with a useful choice.

All references, including any patents or patent applications, cited in this specification are hereby incorporated by reference. No admission is made that any reference constitutes prior art. The discussion of the reference states what their authors assert, and the applicants reserve the right to challenge the accuracy and pertinency of the cited documents. It will be clearly understood that, although a number of prior art publications are referred to herein, this reference does not constitute an admission that any of these documents forms parts of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description, which is given by way of example only.

### DISCLOSURE OF INVENTION

According to one aspect of the present invention there is provided a method for increasing the copper levels in an animal by the administration of a composition containing copper dextran in a manner selected from parenterally; intramuscularly; and a combination thereof. According to another aspect of the present invention there is provided a method, substantially as described above, in which the copper dextran composition is administered in an aqueous composition.

According to another aspect of the present invention there is provided a method, substantially as described above, in which the concentration of copper dextran in an administered composition is 5±12% of elemental copper, by weight.

According to another aspect of the present invention there is provided a method, substantially as described above, in which for adolescent and adult deer exceeding 60 kg, the administered dose is 120±100 mg of elemental copper.

According to another aspect of the present invention there is provided a method, substantially as described above, in which the dose for cattle and other animal species is proportionally calculated from 50±20 mg of elemental copper per 75 kg of body weight.

According to another aspect of the present invention there is provided a method, substantially as described above, in which the amount of composition containing copper dextran administered is sufficient to result in an increase in blood serum copper concentration of at least 1.5 μmol/l after 24 hours.

According to another aspect of the present invention there is provided a method, substantially as described above, in which the increase in blood serum copper concentration exceeds 2.5 lμmol/l after 24 hours.

According to another aspect of the present invention there is provided a method, substantially as described above, in which the amount of composition containing copper dextran administered is sufficient to raise the blood serum copper concentration above 15.0 μmol/l after 24 hours. According to another aspect of the present invention there is provided a method, substantially as described above, in which the blood serum copper concentration remains above 15.0 μmol/l after 48 hours.

According to another aspect of the present invention there is provided a method, substantially as described above, in which the blood serum copper level is 18.5 μmol/l or greater after 24 hours from administration.

According to another aspect of the present invention there is provided a method, substantially as described above, in which the amount of composition containing copper dextran administered is sufficient to result in an increase in mean liver copper concentration of at least 800 μmol/kg after 14 days.

According to another aspect of the present invention there is provided a method, substantially as described above, in which the increase in mean liver copper concentration exceeds 500 μmol/kg after 28 days.

According to another aspect of the present invention there is provided a method, substantially as described above, in which the amount of composition containing copper dextran administered is sufficient to increase the mean liver copper concentration above 100 μmol/kg after 90 days. According to another aspect of the present invention there is provided a method, substantially as described above, in which the mean liver copper concentration remains above 1000 μmol/kg after 14 days.

According to another aspect of the present invention there is provided a method, substantially as described above, in which the mean liver copper level is 1000 μmol/kg or greater after 14 days from administration.

According to another aspect of the present invention there is provided a method, substantially as described above, in which the amount of composition containing copper dextran administered is an amount insufficient to lead to abscess formation.

According to another aspect of the present invention there is provided a method, substantially as described above, in which the calculated dose for administration is split into two or more portions and administered at separate sites either substantially concurrently, or sequentially within a period of 72 hours, more preferably 48 hours and most preferably within 24 hours. According to another aspect of the present invention there is provided a method, substantially as described above, in which the an animal to which the composition containing copper dextran is administered are deer.

According to another aspect of the present invention there is provided a method, substantially as described above, in which the composition containing copper dextran is administered to sheep breeds disposed to a copper deficiency and/or to individual sheep with a diagnosed copper deficiency.

According to another aspect of the present invention there is provided a method, substantially as described above, in which the composition containing copper dextran is administered to cattle, goats, and/or sheep.

According to a further aspect of the present invention there is provided a composition containing a copper dextran, in a form suitable for administration to an animal to increase copper levels. According to another aspect of the present invention there is provided a composition, substantially as described above, suitable for parenteral administration.

According to another aspect of the present invention there is provided a composition, substantially as described above, which is substantially aqueous.

According to another aspect of the present invention there is provided a composition, substantially as described above, in which the concentration of copper dextran is within the range of 0.1 through 10.0% inclusive, by weight.

According to another aspect of the present invention there is provided a composition, substantially as described above, when administered to an animal.

According to yet a further aspect of the present invention there is provided a method for the treatment of copper deficiency in cervine species, comprising the intramuscular administration of a composition, substantially as described above.

The present invention makes use of a composition containing copper dextran to introduce copper into an animal's system. Dextrans are complex sugars, which have a capacity to bind minerals and are thought to be ingested by phages in the body where they are metabolised, releasing copper. Iron dextrans have been used for the administration of iron to pigs and consequently the applicants have investigated the suitability of a composition containing copper dextran for introducing copper into an animal's system. A variety of dextran types are included in the term dextrans being, high molecular weight polysaccharides.

Copper dextran, which is understood to contain a chelated form of copper, is water soluble which affords it with some advantages over the prior art compounds. The copper glycinates and edtates are insoluble compounds and are generally in the form of suspensions, emulsions, or pastes. This gives rise to some administration problems including the need for larger bore needles. Aqueous solutions can allow for faster injection times with smaller needles that are less likely to distress the animal.

However, it is also a consideration that an aqueous solution, which can theoretically more quickly interact with tissue at the injection site (as opposed to an insoluble form of copper) can give rise to increased site reactions. However, surprisingly, the intramuscular administration of composition containing copper dextran into deer shows a significantly decreased site reaction as opposed to what would be expected from prior art glycinate and edtate compositions.

Accordingly preferred embodiments of the present invention will comprise compositions containing a copper dextran. Various dextrans are available and thus the ability exists for different copper dextran compounds to be produced. The present invention includes within its scope all such copper dextrans though the currently preferred copper dextran is that manufactured by Dextran Products of Canada.

Preferred compositions for use in the method of the present invention are aqueous or substantially aqueous in character. However, multiple phase compositions and emulsions may also be included. The use of pastes or tableted forms of composition containing copper dextran are also within the scope of the present invention. Such forms may find use for non-parenteral administration, though may also be considered for implanted depots. However the preferred method of administration according to the present invention is the intramuscular administration of a liquid, and preferably aqueous, copper dextran containing compositions.

The quantity and concentration of copper dextran in the various compositions may vary. For an aqueous composition, the preferred concentration is 5±2% by weight, with the percentage representing the amount of elemental copper present in solution. However, in practice, concentrations from 0.01% (elemental copper) up to saturated copper dextran solutions may be used though it is envisaged that concentrations in the 0.5% through 6% range will be most frequently used. Please note that all the ranges given within this specification are inclusive, unless specifically noted otherwise.

In other embodiments, the dextran solution may be combined with a liquid of a different phase and/or a solid material (to form a paste). Materials which are substantially inert with respect to decomposition of the copper dextran should be relied upon. Such other materials should be pharmacologically acceptable, and may comprise or include other compounds which are to be beneficially administered to the animal.

Another variation is to gel aqueous compositions containing copper dextran.

Dosage rates will tend to vary according to the animal and the amount of deficiency. For a known deficiency, dose rates of around 120±100 mg may be considered though it is anticipated that in many instances average doses are likely to fall around 60±30 mg (based on elemental copper). Dosage rates outside of these ranges may be considered and used, though as will be appreciated, the exact dosages for any individual will rely on a variety of factors. Hence these ranges are not intended to be limiting, but rather representative of useful starting points in determining correct administration levels.

The above doses have been focussed on an adult or adolescent deer weighing 60 kg or greater. For deer, and another an animal, of other weights then the amount administered may be reduced or increased accordingly. As a general guideline the broader range of 120±10 mg, or narrower dosage rate of 60±30 mg (based on elemental copper) per 75 kg of body weight may be used as a guide or starting point. The dosage rate may be adjusted proportionally according to the exact body weight.

Another consideration is the severity of the deficiency. Where there is a severe deficiency then several separate administrations over an extended period of time may be preferable to a single or short term large dose rate to counter a severe diagnosed deficiency. For particular an animal that are quite sensitive, and which may still react adversely to preferred dosage amounts, then the preferred dose may be split into two or more portions which may be either administered at different sites concurrently (to minimise local site reaction effects), or sequentially over a period of time. If possible (as a general guide) this period of time should be within 72 hours, more preferably within 48 hours, and most preferably within 24 hours. Two methods of measuring copper levels in an animal have been used by the applicant, one in which the blood serum copper concentration is measured and the other where the mean liver copper concentration is measured. It has been found by the applicant that the preferred method of measurement is via mean liver copper concentrations. Blood serum levels give a useful indicator when copper levels are either high or low, however at around standard blood serum copper levels the accuracy of this method is less reliable. By measuring copper levels via mean liver concentrations, it has been found that are more reliable. It will be appreciated by a person skilled in the art that further known alternatives for measuring copper concentrations in an animal are also possible.

It is also noted that the method of the present invention may be used not only on deer, but also on cattle, sheep, and other an animal. Consideration needs to be given to particular breeds—for instance some breeds of sheep such as the Finish Land Race are prone to copper deficiency while other breeds such as the Texel are not. Accordingly, caution should be exercised when dealing with breeds known to have the potential for copper toxicity problems. In such cases some trial and experimentation by the user to determine acceptable dosage rates and dose schedules for copper dextran compositions of the present invention should be undertaken.

### BEST MODES FOR CARRYING OUT THE INVENTION

The present invention may take a number of different forms and it is envisaged that there will be a number of varying embodiments for the compositions, as well as how these are administered to increase copper levels in an animal. By way of example some test data from trials on red deer are included.

Example 1 compares the sub-cutaneous to intra muscular (currently preferred method) administration of a composition containing copper dextran in an aqueous solution. This example showed a measurable increase in blood copper serum levels indicating that the administration of the composition was an active and useful method of raising copper levels. Example 2 trials two compositions containing copper dextran, a standard dose and a high dose for safety, toxicity and efficacy in farmed red deer. The results are compared to a control group of untreated deer. Liver copper concentrations are measured rather than blood serum concentrations. The results showed a significant increase in liver copper concentrations for the first 90 days from administration. A reduction close to normal levels was shown after 168 days.

### Example 1

### Materials and Methods

Six one-year-old red deer females grazing pasture were chosen for this study.

### Design

Six deer were randomly allocated to subcutaneous (n=3) or intramuscular (n=3) injection groups.
Temperatures were measured rectally. Blood samples were collected by jugular venepuncture into plain vacutainer tubes using a 1″×20 gauge draw-off needle.
Injection sites were examined as follows:
   visually
   by palpation running fingertips across the injection site
   by gathering skin and subcutaneous tissue between thumb and forefinger to detect subcutaneous tissue reactions or oedema
   by deep muscle palpation applying pressure to the injection site to palpate tissue swellings or nodules
   comparing tissue depth on the side of injection with the opposite side of the neck.
Injection site was into the dorsolateral aspect of the anterior neck approximately 10 cm caudal to the ear.

### Test Product

A 5% copper dextran aqueous solution supplied by Dextran Products of Canada on request.

### Procedures

### Time 0

Temperature
Blood sample for GLDH, GGT, AST and serum copper
Animal restraint
   For injection, an animal were restrained in a pneumatic deer restraining device and the head was physically restrained to ensure accurate placement of the test product.
Site preparation
   The injection site was surgically clipped using a No. 40 blade to a size approximately 8 cm×8 cm. The injection site was palpated as above to ensure there were no non-specific lesions. The injection site was then swabbed with methylated spirits using cotton wool.
Injection
   A sterile 2 ml syringe and a new sterile 20 g×1″ needle was used for each animal.
(a) Subcutaneous injection
   The skin at the centre of the prepared site was grasped between thumb and forefinger and the needle inserted subcutaneously under the thumb. 1 ml of the test product was injected.
   Visual observation was used to ensure that all product was administered subcutaneously.
(b) Intramuscular administration
   The needle was inserted perpendicular to the skin to a depth of approximately 2 cm into the muscle and 1 ml of the test product injected.
   Deer were then released to pasture.

### 6 Hours

Deer were returned from pasture and rectal temperatures taken, injection sites monitored and blood samples collected for copper and GLDH as above.

### 24 Hours

Deer were returned from pasture and rectal temperatures taken, injection sites monitored and blood samples collected for copper and GLDH as above.

### 48 Hours

Deer were returned from pasture and rectal temperatures taken, injection sites monitored and blood samples collected for copper as above.

### 5 Days

Deer were returned from pasture and rectal temperatures taken, injection sites monitored and blood samples collected for copper as above.
While the protocol provided by Bomac Laboratories Ltd concluded at 5 days, there were tissue reactions significant enough in one of the subcutaneous an animal and a further animal described below, to warrant continued investigation as follows:

### Day 7

Site observation

### Day 12

Site observation

### Day 17

Site observation

### Results

Note: One deer (836) struggled during subcutaneously administration. A suspicion arose that some product may have been injected intradermally so this animal was replaced for sampling. However, this animal was retained to provide additional observations of injection site lesions where incorrect or doubtful injection placement occurred.

### Injection Procedure

The test product was of low viscosity and was easily administered without indication of tissue irritation or pain associated with the product.

### Temperatures

Temperatures are presented in Table I.

TABLE I
Temperatures (° C.)

| | | Time | | | | |
|---|---|---|---|---|---|---|
| Route | ID | 0 h | 6 h | 24 h | 48 h | 5 days |
| Subcutaneous | 846 | 40.8 | 40.9 | 40.4 | 39.9 | 39.4 |
| | 851 | 40.5 | 40.3 | 39.5 | 39.2 | 39.9 |
| | 953 | 40.0 | 41.1 | 40.0 | 39.5 | 39.7 |
| Intramuscular | 845 | 40.4 | 40.9 | 40.6 | 39.6 | 39.4 |
| | 849 | 40.6 | 39.8 | 39.6 | 39.5 | 39.2 |
| | 857 | 40.8 | 40.3 | 40.0 | 40.0 | 39.9 |

At the time of copper administration temperatures were uniformly high. Generally as the trial progressed temperatures fell towards the normal resting range. The explanation for this temperature pattern is that it is normal for small groups of deer to become agitated and for them to be more active in the mustering phase into the yard. It was noted that at each sampling period the deer were progressively more relaxed.

This pattern of body temperatures coupled with knowledge of nommal patterns of deer temperatures suggests that the injectable copper formulation has had no significant influence on body temperature.

### Serum Copper

Serum copper results are presented in Table II.

TABLE II
Serum copper concentration (μmol/L)

| | | Time | | | | |
|---|---|---|---|---|---|---|
| Route | ID | 0 h | 6 h | 24 h | 48 h | 5 days |
| Subcutaneous | 846 | 15.2 | 18.4 | 17.5 | 16.9 | 19.8 |
| | 851 | 17.5 | 19.5 | 19.8 | 18.8 | 20.8 |
| | 853 | 22.6 | 26.8 | 25.6 | 20.5 | 26.8 |
| Mean | | 18.4 | 21.5 | 21.3 | 18.7 | 22.5 |
| Intramuscular | 845 | 15.0 | 17.2 | 17.8 | 16.4 | 17.3 |
| | 849 | 17.7 | 20.2 | 21.1 | 19.6 | 21.0 |
| | 857 | 14.9 | 16.0 | 16.6 | 19.5 | 17.1 |
| Mean | | 15.9 | 17.8 | 18.5 | 18.5 | 18.5 |

Serum copper concentrations at time 0 were in the high normal range (8-22 μmol/l) with 853 being narrowly above the normal range.

On average, the mean blood copper concentration increased by approximately 2-3 μmol/L within 6 hours. Mean copper concentration at 48 hours after subcutaneous injection had fallen to the pre-injection concentration, but at 5 days had increased by 4 μmol/l. Conversely, mean blood copper concentration after intramuscular injection increased to 18.5 μmol/l and remained constant. While these numbers are low, data suggests that copper was being absorbed from injection sites.

TABLE III
Blood GLDH concentrations

| | | Time | | |
|---|---|---|---|---|
| Route | ID | 0 h | 6 h | 24 h |
| Subcutaneous | 846 | 5 | 4 | 5 |
| | 851 | 5 | 3 | 2 |
| | 853 | 2 | 3 | 2 |
| Intramuscular | 845 | 1 | 4 | 1 |
| | 849 | 3 | 4 | 3 |
| | 857 | 2 | 4 | 4 |

All GLDH concentrations fall within normal range.

TABLE IV
Blood AST (IU/L) and GGT (IU/L) concentrations
prior to injection

| ID | AST | GGT |
|---|---|---|
| 845 | 75 | 52 |
| 846 | 280 | 64 |
| 849 | 104 | 17 |
| 851 | 84 | 22 |
| 853 | 71 | 27 |
| 857 | 78 | 32 |

Animal 846 had AST and GGT concentrations very marginally above the normal range. These were considered to be clinically insignificant.

TABLE V
Liveweight (kg)

| | | Time | | | |
|---|---|---|---|---|---|
| Route | ID | 0 h | 5 days | 12 days | 17 days |
| Subcutaneous | 846 | 78.5 | 78.0 | 80.5 | 82 |
| | 851 | 80.0 | 79.0 | 81.0 | 81 |
| | 853 | 84.0 | 81.5 | 84.0 | 84.5 |
| Mean | | 80.8 | 79.5 | 81.8 | 82.5 |
| Intramuscular | 845 | 79.5 | 79.0 | 81.0 | 81 |
| | 849 | 71.5 | 71.5 | 72.5 | 74 |
| | 857 | 72.0 | 69.5 | 72.0 | 74 |
| Mean | | 74.3 | 73.3 | 75.2 | 76.3 |
| Additional hind | 836* | 74 | 73 | 74.5 | 76.5 |

*This is the hind which had uncertain placement of test product, and in which the skin sloughed severely.

TABLE VI
Description of tissue lesions at injection sites

| Route | ID | 0 h | 6 h | 24 h | 48 h | 5 days | 7 days | 12 days | 17 days |
|---|---|---|---|---|---|---|---|---|---|
| Subcutaneous | 846 | N | N | Slight | Firm subcut. | Firm subcut. | Firm oedema 6 × | Fibrous swelling. | Fibrous swelling |
| | | | | oedema . | Oedema, 8 cm | oedema 7 × 3 cm. | 2.5 cm. Becoming | same size. small | contracting. 2.0 × 5 |
| | | | | 0.5 mm | long, 4 cm wide. | 1.5 cm when | fibrous 2 cm when | superficial slough | cm. Skin still crusty. |
| | | | | subcut, | Gravitating. 1.5 | grasped | grasped. Skin | | Superficial slough |
| | | | | 3 cm | cm thick when | | feels dry and | 1 cm × 0.5 cm at | approx. 0.5 × 1 cm. |
| | | | | diameter | grasped | | crusty | injection site | |
| | 851 | N | N | N | N | N | N | N | N |
| | 853 | N | N | N | 4 cm diffuse | 2.5 cm diam. | 2.5 cm diam. firm | N | N |
| | | | | | subcut oedema. . 1 | diffuse oedema . | oedema . 1.5 cm | | |
| | | | | | cm when grasped | 1.5 cm when | when grasped | | |
| | | | | | | grasped | | | |
| Intramuscular | 845 | N | N | N | N | N | N | N | N |
| | 849 | N | N | N | N | N | N | N | N |
| | 859 | N | N | N | N | N | N | N | N |
| Additional* | 836 | N | N | N | 3 cm × 6 cm | 3 × 6 firm | Hard swelling 1 | Skin sloughing | Borders of |
| | | | | | subcut. oedema. | oedema. Slight | cm deep 6 × 8 cm. | with exudate. | sloughing tissue dry. |
| | | | | | Gravitating . 1 cm | serum ooze from | Skin feels dry and | Oval dry dead | Wound healing. |
| | | | | | when grasped | injection site. . 2 | crusty | skin 6 cm × 3 cm | Central oval dead |
| | | | | | | cm when grasped | | | skin not yet |
| | | | | | | | | | sloughed. Wound |
| | | | | | | | | | contracting |

N = No visible or palpable reaction
*= Deleted from study because of uncertainly of injection placement, but retained to provide additional observation of tissue reactions

### Observations

1. The test product was easy to administer and there was no obvious observable pain response to administration.
2. There were no blood biochemical, clinical or bodyweight effects of significance.
3. Blood copper levels were elevated after both intramuscular and subcutaneous routes of administration.
4. No visible or palpable lesions were observed after intramuscular administration.
5. Two of 3 deer showed injection site lesions after subcutaneous administration.
   One mild, transitory oedema
   One longer standing larger oedematous lesions which became fibrous and from which superficial skin sloughing occurred.
6. One deer with uncertain placement of subcutaneous injection, with some product possibly intradermal, showed a severe tissue reaction and skin sloughing.

### Example 2

A composition containing copper dextran was evaluated for safety, toxicity and efficacy in farmed red deer.

Twelve deer were treated by intramuscular injection of a dose of 1 ml (50 mg copper) for & month-old deer and 2 ml (100 mg copper) for 18-month-old deer. This was used as a standard dose. The second group received three times that dose rate. At days 90 and 168, an untreated control group of equivalent deer were compared as a control.

Liver biopsies were collected prior to administration and at 14, 28, 90 and 168 days thereafter. At 90 and 168 days a similar group of untreated deer were also sampled in a similar manner as controls.

### Results:

The results found were as follows:

TABLE VII
Mean liver copper concentrations [μmol/kg]

| | Number of Days | | | | |
|---|---|---|---|---|---|
| Dose | 0 | 14 | 28 | 90 | 168 |
| Standard | 200 | 1052 | 780 | 306 | 135 |
| High | 158 | 1918 | 1332 | 633 | 263 |
| Control | | | | 93 | 107 |

After 90 days the standard and high dose treated groups still had significantly different liver copper concentrations, being approximately 3 and 7 times higher, respectively, than those of controls on that day. At day 168, there was no difference in mean liver copper concentration between treatment groups, but the mean of the high treatment group was significantly higher that the untreated controls.

It is concluded that a composition containing copper dextran is safe and efficacious at both dose rates in deer with pre-injection concentrations ranging from 84 to 570 μmol/kg.

Aspects of the present invention have been described by way of example only and it should be appreciated that modifications and additions may be made thereto without departing from the scope thereof as defined in the appended claims.

1. A method for increasing the copper levels in an animal by the administration of a composition containing copper dextran in a dose in a manner selected from parenterally; intramuscularly; and a combination thereof.

2. A method for increasing the copper levels in an animal as claimed in claim 1 in which the concentration of copper dextran in the composition is 5±2% of elemental copper in the composition, by weight.

3. A method for increasing the copper levels in an animal as claimed in claim 1 in which for adolescent and adult deer exceeding 60 kg, the administered dose is 120±100 mg of elemental copper in the composition, by weight.

4. A method for increasing the copper levels in an animal as claimed in claim 1 in which the dose of the composition is an amount from 50±20 mg of elemental copper per 75 kg of body weight.

5. A method for increasing the copper levels in an animal as claimed in claim 1 in which the composition contains 60±30 mg of elemental copper per 75 kg of body weight.

6. A method for increasing the copper levels in an animal as claimed in any one of the above claims in which the dose is sufficient to result in an increase in blood serum copper concentration in the animal of at least 1.5 μmol/l 24 hours after administration.

7. A method for increasing the copper levels in an animal as claimed in either claim 1 or claim 6 in which the increase in blood serum copper concentration in the animal exceeds 2.5 μmol/124 hours after administration.

8. A method for increasing the copper levels in an animal as claimed in either claim 1, claim 6 or claim 7 in which the amount of composition administered is sufficient to raise the blood serum copper concentration in the animal above 15.0 μmol/l 24 hours after administration.

9. A method for increasing the copper levels in an animal as claimed in either claim 1 or claims 6 to 8 the above claims in which the blood serum copper concentration in the animal remains above 15.0 μmol/l after 48 hours from administration.

10. A method for increasing the copper levels in an animal as claimed in either claim 1 or claims 6 to 9 the above claims in which the blood serum copper level in the animal is above 18.5 μmol/l after 24 hours from administration.

11. A method for increasing the copper levels in an animal as claimed in any one of the above claims in which the amount of composition administered is sufficient to result in an increase in mean liver copper concentration of the animal exceeds 800 μmol/kg after 14 days.

12. A method for increasing the copper levels in an animal as claimed in any one of the above claims in which the amount of composition administered is sufficient to increase the mean liver copper concentration of the animal exceeds 500 μmol/kg after 28 days.

13. A method for increasing the copper levels in an animal as claimed in any one of the above claims in which the amount of composition administered is sufficient to increase the mean liver copper concentration of the animal exceeds 100 μmol/kg after 90 days.

14. A method for increasing the copper levels in an animal as claimed in any one of the above claims in which the amount of composition administered is sufficient to increase the mean liver copper concentration in the animal remains above 1000 μmol/kg after 14 days.

15. A method for increasing the copper levels in an animal as claimed in any one of the above claims in which the amount of composition administered is sufficient to increase the mean liver copper level in the animal is 1000 μmol/kg or greater after 14 days from administration.

16. A method for increasing the copper levels in an animal as claimed in any one of the above claims, in which the dose is split into two or more portions and administered at separate sites on the animal in a manner selected from: substantially concurrently, and sequentially.

17. A method for increasing the copper levels in an animal as claimed in claim 16, wherein the time period is, within a period of 24 hours, 48 hours or 72 hours.

18. A method for increasing the copper levels in an animal as claimed in any one of the above claims in which the animal is a deer.

19. A method for increasing the copper levels in an animal as claimed in any one of claims 1 to 17 in which the animal is a sheep breed disposed to a copper deficiency or an individual sheep with a diagnosed copper deficiency.

20. A method for increasing the copper levels in an animal as claimed in any one of claims 1 to 17 in which the animal is selected from cattle, goats, and sheep.

21. A composition containing copper dextran in a form suitable for administration to an animal for use to increase copper levels in the animal.

22. A composition containing copper dextran for use in the method as claimed in any one of claims 1 to 20.

23. A composition containing copper dextran as claimed in either claim 21 or 22, in which the form of the composition is selected from a substantially aqueous solution or suspension, a paste, a tablet form, and a combination thereof.

24. A composition containing copper dextran, as claimed in any one of claims 21 to 23 in which the concentration of copper dextran is within the range of 0.1 through to 10% inclusive by weight of composition.

25. A composition containing copper dextran as claimed in claim 24 in which the concentration of copper dextran is within the range of 0.5 through 6.0% inclusive by weight.

26. A composition containing copper dextran as claimed in any one of claims 21 to 23, in combination with at least one other material selected from the group: a material that is substantially inert with respect to decomposition of the copper dextran; pharmacologically acceptable other material; and a combination thereof.

27. A method for the treatment of copper deficiency in an animal species, comprising the intramuscular administration of a composition as claimed in claims 21 to 26.

28. A method for the treatment of copper deficiency in an animal species as claimed in claim 27 in which the animal is a deer.

29. A method for increasing the copper levels in an animal as claimed in claims 1-20 substantially as hereinbefore described and with reference to the examples.

30. A composition containing copper dextran as claimed in claims 21-26 substantially as hereinbefore described and with reference to the examples.

31. A method for the treatment of copper deficiency in cervine species, as claimed in either claim 27 or 28 substantially as herein described and with reference to the examples.
